(19) United States
(12) Patent Application Publication
(10) Pub. No.: US 2022/0265479 A1
(43) Pub. Date: Aug. 25, 2022

(54) SUPPORT PORTION-BONDED MEDICAL TAPE

(71) Applicant: YUGENKAISHA CHOURYU, Shizuoka (JP)

(72) Inventor: Toshifumi HIRASHIMA, Shizuoka (JP)

(21) Appl. No.: 17/594,003

(22) PCT Filed: Aug. 20, 2021
(86) PCT No.: PCT/JP2021/030551

§ 371 Date: Sep. 30, 2021

(30) Foreign Application Priority Data
Sep. 17, 2020 (JP) 2020-156707

(51) Int. Cl. A61F 13/02

(52) U.S. Cl. CPC A61F 13/0226; A61F 13/0253

(57) ABSTRACT

There is provided a support portion-bonded medical tape that has a high effect of alleviating a persistent skin irritation during an application duration caused by a residual shrink force and aims to be applied on a skin that exhibits an excessive reaction even when a degree of residual shrink force as a cause is an insignificant irritation so as to be described as of a level of magnifying lens or microscope, not as of a level of naked eye.

A support portion-bonded medical tape of the present invention includes an elastic base member portion film-formed of a solution polymer and having an elastic function, an adhesion portion having a function to apply and hold the elastic base member portion on a skin, a release portion having a function to protect the adhesion portion, and a support portion having a function to support the elastic base member portion. The support portion, the elastic base member portion, the adhesion portion, and the release portion are provided in this order from an upper layer. In a manufacturing process, the solution polymer used as a material for the elastic base member portion is applied to an upper layer of the adhesion portion, and the support portion is laminated on an upper layer of the elastic base member portion before or after completion of forming shrinkage. A residual shrink force existing in the support portion-bonded medical tape is reduced by removing the release portion at a time of use.

### TECHNICAL FIELD

The present invention relates to a support portion-bonded medical tape.

### BACKGROUND ART

Conventionally, for medical industries or the like, a medical tape that uses a film material, such as a polyurethane film, having a function excellent in waterproof performance, moisture permeability, skin followability, and the like has been selected in order to cover and fix a gauze and a pad, fix a surgical tool, such as an indwelling needle or a catheter, cover and protect a wound or a skin, and the like. The indwelling needle is an injection needle used by being inserted into a vein and fixed to a body for blood sampling or drip infusion. The indwelling needle is also used in drip infusion or the like used for about a week.

Since these film materials have a very thin thickness of approximately 10 μm, it cannot independently hold a planar shape and is easily rounded under a natural environment. For this reason, as a support for holding the planar shape, a shape protection cover having stiffness higher than that of an elastic base member of the film material, such as an inelastic plastic film, is laminated with the film material in order to hold the planar shape and prevent formation of wrinkles or the like.

Note that, in its manufacturing process, as means to laminate the support for holding the planar shape and the film material, there has been widely used a method in which, after applying a mixed liquid (a urethane resin liquid and a crosslinking agent liquid) that serves as a raw material of the polyurethane film on an upper layer (one surface) of the inelastic plastic film, the mixed liquid is hardened to form a film, and thus, the polyurethane film is obtained, and the support for holding the planar shape and the film material are laminated.

However, since the above-described method simultaneously performs the film formation of the polyurethane film and the lamination on the support, there is generated a shrink force (an internal stress) remaining due to a forming shrinkage associated with the hardening of the mixed liquid serving as the raw material of the polyurethane film in the polyurethane film. Hereinafter, the “shrink force remaining in the elastic base member during the manufacturing process” will be referred to as a “residual shrink force.” Accordingly, in the medical tape that uses the polyurethane film obtained in the above-described method as the elastic base member of the medical tape and the inelastic plastic film as the support of the medical tape, that is, “the medical tape formed by simultaneously performing the film formation of the elastic base member in a film form and the lamination on the support positioned on an upper layer using the mixed liquid (the urethane resin liquid and the crosslinking agent liquid) as a raw material serving as the raw material of the polyurethane film, applying an adhesion on a lower layer of the elastic base member, providing an inelastic release sheet on the lowermost layer, and providing a support for holding the planar shape on an upper layer of the elastic base member”, the residual shrink force associated with the forming shrinkage resides in the film material as the elastic base member. That is, when the elastic base member is not attached on the support, the elastic base member freely shrinks, and thus, the residual shrink force is not generated. Meanwhile, when the support is well attached on the elastic base member, the elastic base member cannot freely shrink except in a film thickness direction, and thus, the residual shrink force is generated near an attachment interface between the elastic base member and the support.

Note that the support-bonded medical tape is applied in the following sequence: 1) remove the release sheet; 2) apply the tape on a skin; and 3) remove the support. In addition, the residual shrink force of the support-bonded medical tape is not eliminated even by entirely removing the release sheet. After the tape is applied on the skin, the residual shrink force is released by removing the support, and acts as a force to continuously shrink the skin. Since the skin is shrunken, the residual shrink force causes a persistent skin irritation during the application duration.

Here, the residual shrink force existing in the support-bonded medical tape will be described with reference to FIGS. 1 and 2. FIG. 1 is an explanatory diagram illustrating a state in which after a support-bonded medical tape having a bisected support structure is applied on a skin, a support of the thumb side is removed. In order to compare states of the elastic base members underlying the support of the thumb side and the support of the fifth finger side, the support of the thumb side is removed, and the support of the fifth finger side is not removed. While fine wrinkles are observed in the elastic base member of the thumb side from which the support is removed after the tape is applied on the skin, no change is observed in the elastic base member of the fifth finger side from which the support is not removed. FIG. 2 illustrates a state in which the remaining support, that is, the support of the fifth finger side is removed. In FIG. 2, fine wrinkles are also observed in the elastic base member of the fifth finger side. It is conceived that this is because, as the support is removed from the support-bonded medical tape, the elastic base member is released from the support and shrunken so as to exhibit fine wrinkles in the elastic base member, and therefore, a residual shrink force associated with the forming shrinkage exists in the elastic base member. In this manner, in the support-bonded medical tape, the residual shrink force is released by removing the support. Therefore, the residual shrink force associated with the forming shrinkage existing in the support-bonded medical tape can be observed by after applying the medical tape on the skin, removing the support to shrink the elastic base member, thereby causing fine wrinkles to appear in the elastic base member.

Note that, in the medical tape industry, a warning, such as “please apply without pulling (without stretching) because it may cause skin irritation,” is called in a handling manual or the like of the product. Therefore, in the medical tape industry, it is inferred that a risk of applying an elastic base member portion of an elastic medical tape on a skin in a “pulled and stretched state” should be sufficiently understood as a common sense of those skilled in the art. Additionally, in the plastic industry, it is conceived to be a common sense of those skilled in the art that applying a solution polymer on an upper layer of an inelastic support (for example, a plastic film), drying the solution polymer and forming a polyurethane film and laminating it on the support at the same time generates the residual shrink force (the internal stress) associated with the forming shrinkage within the polyurethane film.

On the other hand, in the medical tape industry, it is inferred that there is little recognition (a common sense of those skilled in the art) that the residual shrink force associated with the forming shrinkage exits within the polyurethane film as the elastic base member portion of the support-bonded medical tape. If there is the recognition (the common sense of those skilled in the art) of the existence of the residual shrink force associated with the forming shrinkage in the medical tape industry, and it is understood that the residual shrink force has a harmful effect, there should be a description of warning or the like on the “residual shrink force associated with the forming shrinkage existing in the elastic base member portion” in a handling manual or the like of the product. In a handling manual or the like of a medical tape currently manufactured and sold, there is only a warning that says “if a symptom that seems to be a skin disorder (rash, redness, itching, and the like) appears during use of this product, please stop using and carry out an appropriate treatment,” which is a warning that contains a problem of possibly misleading that it is caused by the user's constitutive endogenous cause. Accordingly, there has been a problem that, even though it is a medical tape used by a user with a purpose, the user himself uses the medical tape without recognizing the harmful effect associated with the “residual shrink force associated with the forming shrinkage existing in the elastic base member portion,” and moreover, without any preventive measures. Furthermore, a root of this problem is that there is a contradiction that the “residual shrink force associated with the forming shrinkage” is the common sense of those skilled in the art in the plastic industry but it is not the common sense of those skilled in the art in the medical tape industry where products using similar materials are manufactured and sold, and there has been a problem that no countermeasure has been taken for the residual shrink force associated with the forming shrinkage having the harmful effect in the medical tape industry.

In a part, a medical tape (for example, see Patent Document 1) for alleviating a persistent skin irritation during application duration of an elastic base member of an elastic medical tape has been proposed, and the persistent skin irritation during application duration of the support-bonded medical tape caused by the residual shrink force is improved. However, this medical tape requires to laminate an elastic base member portion and an anti-expansion portion while the elastic base member portion is in a slacked state or in a not-pulled state in the manufacturing process, and therefore, the manufacturing of this medical tape raised a problem in manufacturing equipment, manufacturing cost, and the like from the aspect of efficiency. Accordingly, in the conventional support-bonded medical tape using inelastic material as the support, the production that deals with the residual shrink force associated with the forming shrinkage is not easy, but generates the residual shrink force associated with the forming shrinkage in the elastic base member portion of the medical tape. Thus, it is not easy to alleviate the persistent skin irritation during application duration caused by the residual shrink force associated with the forming shrinkage, and the current situation is that there has been no means that can appropriately solve such a disadvantage.

### CITATION LIST

[Patent Document]
Patent Document 1: Japanese Patent No. 6312915

### SUMMARY OF INVENTION

In view of the aforementioned problems, it is therefore an object of the present invention to provide a support-bonded medical tape that has a high effect of alleviating a persistent skin irritation during an application duration caused by a residual shrink force associated with a forming shrinkage of, in particular, a support-bonded medical tape and aims to be applied on a skin that exhibits an excessive reaction even when a degree of residual shrink force associated with the forming shrinkage as a cause is an insignificant irritation so as to be described as of a level of magnifying lens or microscope, not as of a level of naked eye.

In order to address the aforementioned problems, the inventor made diligent efforts over and over and completed the present invention. That is, a support portion-bonded medical tape of the present invention includes an elastic base member portion film-formed of a solution polymer and having an elastic function, an adhesion portion having a function to apply and hold the elastic base member portion on a skin, a release portion having a function to protect the adhesion portion, and a support portion having a function to support the elastic base member portion. The support portion, the elastic base member portion, the adhesion portion, and the release portion are provided in this order from an upper layer. In a manufacturing process, the solution polymer used as a material for the elastic base member portion is applied on an upper layer of the adhesion portion, and the support portion is laminated on an upper layer of the elastic base member portion before or after completion of forming shrinkage. A residual shrink force existing in the support portion-bonded medical tape is reduced by removing the release portion at a time of use.

### Advantageous Effects of Invention

The medical tape of the present invention can reduce the residual shrink force twice when the support portion-bonded medical tape is manufactured and when it is used by introducing as a function to a medical tape a means for reducing a force to continuously shrink the skin caused by the residual shrink force of the conventional support-bonded medical tape. The medical tape of the present invention can reduce the residual shrink force of the support portion-bonded medical tape by the lamination at the time of manufacturing, and further can reduce the residual shrink force of the support portion-bonded medical tape again before being applied on the skin. Since the medical tape of the present invention can be applied on the skin in a state in which the residual shrink force is reduced, it can reduce the force to continuously shrink the skin due to the residual shrink force of the support portion-bonded medical tape. Accordingly, it is possible to alleviate a persistent skin irritation during an application duration caused by the residual shrink force. This ensures an application on the skin that exhibits an excessive reaction even when a degree of residual shrink force associated with the forming shrinkage as a cause is an insignificant irritation so as to be described as of a level of magnifying lens or microscope, not as of a level of naked eye, thereby ensuring providing a medical tape, an adhesive skin patch, and the like of low irritation. Furthermore, it is possible to prevent inflammation in sebaceous glands or sweat glands, inflammation in the root of hair or its surrounding tissues, a medical instrument pressure-related wound caused by poor blood circulation in skin cells, capillary vessels, or the like due to pressure, and the like. With the medical tape manufacturing method of the present invention, the medical tape of the present invention can be efficiently manufactured.

### BRIEF DESCRIPTION OF DRAWINGS

FIG. 1 is an explanatory diagram illustrating a state in which a support of a thumb side used for checking the residual shrink force described in paragraph [0007] is removed.

FIG. 2 is an explanatory diagram illustrating a state in which a support of a fifth finger side as a remaining support in the explanatory diagram illustrated in FIG. 1 is removed.

FIG. 3 is a drawing illustrating a manufacturing process of a medical tape according to a first embodiment of the present invention and is a schematic cross-sectional view illustrating a state of a lamination process of a release portion, an adhesion portion and an elastic base member portion.

FIG. 4 is a schematic cross-sectional view illustrating a medical tape according to a first embodiment of the present invention.

FIG. 5 is a schematic cross-sectional view illustrating a state when in use of the medical tape according to the first embodiment of the present invention, and illustrates a state where a release portion 4 is removed, a residual shrink force existing in an elastic base member portion 1 is released, and the elastic base member portion and the adhesion portion have the same length as a support portion.

FIG. 6 is a schematic cross-sectional view illustrating the state when in use of the medical tape according to the first embodiment of the present invention, and illustrates a state where the medical tape is applied on a skin and a support portion 3 is removed in the state illustrated in FIG. 5.

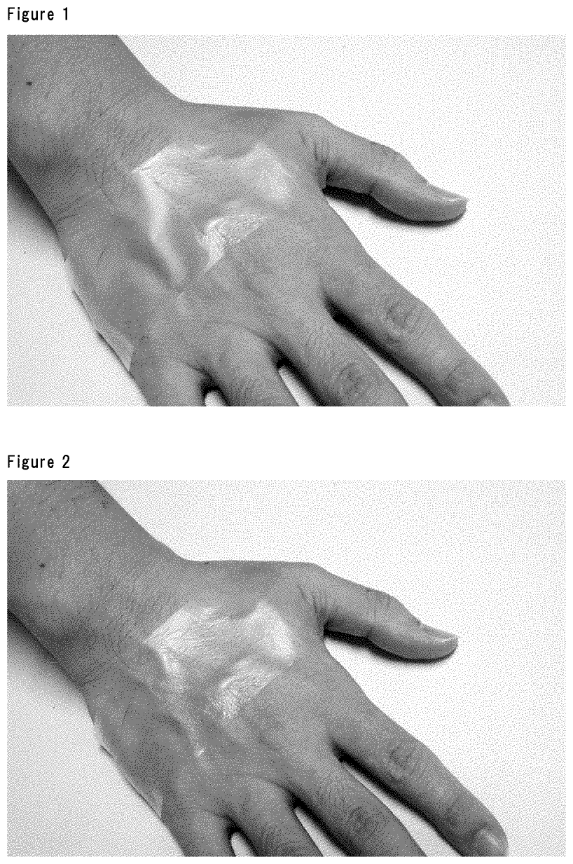

### DESCRIPTION OF EMBODIMENTS

In the course of diligent studies, the inventors found that a cause of a residual shrink force in a support-bonded medical tape in the prior art resided in a forming shrinkage during manufacturing thereof, and the residual shrink force of the support-bonded medical tape in the prior art was not eliminated even after a whole release sheet was removed, but after the medical tape was applied on a skin, the residual shrink force was released by removing the support to become a force that continuously shrinks the skin to shrink the skin, thereby causing a persistent skin irritation during application duration by the support-bonded medical tape. Therefore, the inventor focused on, in the manufacturing process of the support-bonded medical tape, means that reduces the residual shrink force associated with the forming shrinkage by dealing with the residual shrink force associated with the forming shrinkage caused by simultaneously performing a film formation of a polyurethane film and the lamination with the support from a manufacturing stage and when used.

Here, a skin irritation when the support-bonded medical tape in the prior art is used for the purpose to, for example, cover and protect wounds or skin will be described. The skin surface has openings of sebaceous glands or sweat glands and also hair. For this reason, the residual shrink force of the support-bonded medical tape continuously deforms the openings of sebaceous glands or sweat glands and occasionally acts as an occluding force so as to generate inflammation (redness, swelling, fever, pain, and dysfunction) in sebaceous glands or sweat glands in some cases. In addition, the continuous skin shrinking force acts as a force to shrink a skin and persistently draw up the hair, so that inflammation occurs in the root of hair or its surrounding tissues in some cases.

Next, a skin irritation when the support-bonded medical tape is used for the purpose to cover and fix a gauze, a pad and the like or fix a surgical tool, such as an indwelling needle or a catheter, will be described. The residual shrink force of the support-bonded medical tape not only acts to fix the indwelling needle, the catheter, or the like but also acts as a force to persistently press a skin at the portion where the indwelling needle, the catheter, or the like is fixed. The residual shrink force is converted into a force of persistently pressing skin cells, capillary vessels, or the like via such surgical tools.

Next, the force of persistently pressing skin cells, capillary vessels, or the like will be described. For example, the blood vessels are stretched all over a human body, and its total extension length is approximately 100,000 km, which reaches around two and a half laps of the earth. In addition, nearly 95% of the blood vessels are capillary vessels. The capillary vessels have a diameter of approximately 7 μm and a wall thickness of 1 μm or smaller, which is significantly thin. For this reason, even for an unaware insignificant pressure, the capillary vessels of skin may be easily deformed or obstructed.

There are approximately five million red corpuscles, used to carry oxygen to cells over the entire body, per a microliter of blood. The red corpuscles are disk-shaped solid materials having recessed centers on both sides with a diameter of approximately 7 to 8 μm and a thickness of approximately 2 μm. The red corpuscles pass through the capillary vessels having a diameter of approximately 7 μm while they are deformed. However, since deformation of the red corpuscles as solid materials is limited, it is difficult to allow the red corpuscles to pass through the capillary vessels even when slight deformation occurs in capillary vessels. In addition, the capillary vessels may be clogged due to occlusion of red corpuscles in some cases. As a result, oxygen deficiency often occurs in cells due to poor blood circulation.

Poor blood circulation caused by pressure may generate a disease in which tissues or cells of skin locally die, such as a decubitus, so called a bedsore. Experimentally, it is said that a decubitus is generated if a persistent pressure is applied to a same part of a body for two hours or longer. For this reason, in order to prevent the decubitus from occurring in the medical industry, it is recommended that a posture be changed at approximately two hour intervals for a bedridden patient who is in a state of lying on a bed or the like, and at approximately thirty minute intervals for a patient who in a state of sitting on a wheelchair or the like. In this manner, the decubitus is generated due to poor blood circulation caused by pressure for several hours.

Furthermore, the recent support-bonded medical tapes have been remarkably improved in skin followability, moisture permeability, waterproof property, skin adherence, and the like, compared with the conventional elastic medical tapes, so that they can be continuously bonded for about a week. For this reason, the support-bonded medical tapes are used in many cases to cover and fix a gauze, a pad, and the like, and in particular, fix an indwelling needle for drip infusion or the like for about a week. The force of persistently shrinking skin by covering the surgical tool acts as a force to persistently press a portion of the skin where a medical instrument, such as the indwelling needle or the catheter, is fixed for several days. Therefore, a medical instrument pressure-related wound and the like may occur in the fixed portion. This is inferred to be a period of time far exceeding a limitation of several hours for preventing the decubitus from occurring.

Due to improved qualities of adhesives and elastic base members, a long-time fixation with peel-off durability has been possible, and there has been a product that can be attached for a week or longer. In such a product less likely to be peeled off and excellent for long-time fixation, it is important to inform a warning “when removing the product, please gently remove the product along hair streaks so as not to harm skin” in the handling manual or the like. In order to alleviate skin irritation at the time of removing the product, means that does not forcibly peel the product but leaves it until it peels off is effective. However, use of the means that leaves the product until it peels off naturally increases the application duration, and the persistent skin irritation during the application duration caused by the residual shrink force continuously increases.

Note that, even for insignificant irritation, many patients complain of discomfort and distress. However, if insignificant irritation is persistently applied to the body, “hypaesthesia” which causes dull sensation to a stimulus occurs, so that the number of patients who complain of discomfort or distress is reduced. Although this is generally called “habituation,” it does not mean that the stimulus to the body has disappeared, and deformation or occlusion generated in capillary vessels of skin, poor blood circulation, and the like are improved. Even though a degree of the residual shrink force associated with the forming shrinkage described in the paragraphs [0017] to [0023] is an insignificant irritation that may be described as of a level of magnifying lens or microscope, not as of a level of naked eye, it can sufficiently be such a cause.

In this manner, in order to alleviate the persistent skin irritation during the application duration, it is important to take the process that leads to the forming shrinkage into consideration, laminate the medical tape such that the residual shrink force is not generated when manufacturing the support portion-bonded medical tape, and furthermore, when in use of the support portion-bonded medical tape, reduce the force that continuously shrinks the skin due to the residual shrink force associated with the forming shrinkage by releasing the residual shrink force before the medical tape is applied on the skin. Here, in the manufacturing process of the support portion-bonded medical tape, the adhesion portion is provided on an upper layer of the release portion, a solution polymer used as a material of the elastic base member portion is applied on an upper layer of the adhesion portion, and then a film is formed. At this time, the elastic base member portion has a cross-sectional surface in a trapezoidal shape with an upper base shorter than a lower base in association with the forming shrinkage of the solution polymer. In the trapezoidal elastic base member portion, the residual shrink force associated with the forming shrinkage of the solution polymer is generated on the lower base of the elastic base member portion attached on the adhesion portion. On the other hand, since the upper base of the elastic base member portion is not laminated at this moment, it is not attached and shrinks freely, and the residual shrink force is in a state of decreasing from the lower base of the elastic base member portion attached on the adhesion portion toward the upper base of the elastic base member portion in a film thickness direction. Therefore, since there is a difference in the residual shrink force associated with the forming shrinkage of the solution polymer between the lower base of the elastic base member portion in contact with the adhesion portion and the upper base of the elastic base member portion, there is a difference in length and the elastic base member portion has a trapezoidal shape. The residual shrink force on the upper base of the elastic base member portion can be said to be reduced as compared with that on the lower base of the elastic base member portion in contact with the adhesion portion. Then, before or after completion of forming shrinkage of the solution polymer, the support portion is laminated on the upper base of the elastic base member portion, and the support portion-bonded medical tape is completed.

The support portion-bonded medical tape manufactured as described above is used in the following sequence: 1) remove the release portion; 2) apply the tape on a skin; and 3) remove the support portion. In the support portion-bonded medical tape, the release portion is first removed. Then, the elastic base member portion and the adhesion portion of the support portion-bonded medical tape are shrunk. This is because the adhesion portion follows the elasticity of the elastic base member portion by removing the release portion, the residual shrink force generated on the lower base of the elastic base member portion that is attached to the adhesion portion is released, and the residual shrink force existing in the elastic base member portion is reduced. The length of the elastic base member portion and the adhesion portion of the support portion-bonded medical tape shrinks and shortens for the amount of the residual shrink force that has been released. Then, the tape is applied on the skin and the support portion is removed. The support portion and the upper base of the elastic base member portion are laminated before or after the completion of the forming shrinkage of the solution polymer. Thus, the upper base of the elastic base member portion attached to the support portion is in a state where the residual shrink force associated with the forming shrinkage of the solution polymer is reduced. Accordingly, removal of the support portion does not result in an apparent change of shrinkage of the elastic base member portion. Removing the release portion of the support portion-bonded medical tape before it is applied on the skin allows an additional reduction of the residue shrink force of the support portion-bonded medical tape, whereby the tape can be applied on the skin with the residual shrink force reduced. Thus, the reduction of the residual shrink force of the support portion-bonded medical tape twice when it is manufactured and when it is used becomes an effective means to reduce the force that continuously shrinks the skin caused by the residual shrink force of the conventional support-bonded medical tape and reduce persistent skin irritation during the application duration.

Introducing these effective means to the support portion-bonded medical tape as a function ensures applying the medical tape on a skin with the residual shrink force of the medical tape being reduced when in use of the support portion-bonded medical tape, reducing the force that continuously shrinks the skin due to the residual shrink force of the support portion-bonded medical tape, and thus, alleviating the persistent skin irritation during the application duration caused by the residual shrink force, thereby ensuring an application on a skin that exhibits an excessive reaction even with an insignificant irritation. Furthermore, it can be expected that a skin trouble, a medical instrument pressure-related wound, or the like caused by the persistent skin irritation during the application duration is prevented. From this viewpoint, it would be recognized that the conventional support-bonded medical tape fail to consider the residual shrink force associated with the forming shrinkage of the support-bonded medical tape.

Therefore, in order to provide a support portion-bonded medical tape introducing a function to reduce the residual shrink force of the conventional support-bonded medical tape, as a material of the elastic base member portion, the inventor selected a material that could be laminated with the support portion from materials that hardened and formed a film after being applied on the upper layer of the adhesion portion, and furthermore, selected a material usable for medical treatment and excellent in waterproof performance, moisture permeability, skin followability, and the like with elasticity after the film formation. Next, as a material of the adhesion portion, the inventor selected a medical adhesive that had a function to follow the elastic function of the elastic base member portion and caused little adhesive residue, keratin damage or the like at the time of removal. In addition, as a material of the support portion, the inventor selected an inelastic material having a function to support the elastic base member portion, and furthermore, as a material of the release portion, the inventor selected a material having a function to protect the adhesion portion. Thus, the inventor conceived of configuring the support portion-bonded medical tape using the functions included in the respective materials.

By combining these materials, in the manufacturing process of the support portion-bonded medical tape, a solution polymer used as a material of the elastic base member portion is applied on the upper layer of the adhesion portion, the support portion is laminated on an upper layer of the elastic base member portion before or after the completion of the forming shrinkage, and the release portion is removed when in use of the support portion-bonded medical tape, thereby reducing the residual shrink force existing in the support portion-bonded medical tape. Accordingly, it is possible to introduce a means to reduce the force that continuously shrinks the skin caused by the residual shrink force of the conventional support-bonded medical tape as a function to the support portion-bonded medical tape. By obtaining knowledge that alleviating the persistent skin irritation during the application duration caused by the residual shrink force ensures providing a medical tape that can be applied on the skin that exhibits an excessive reaction to even an insignificant irritation, the present invention reached the completion.

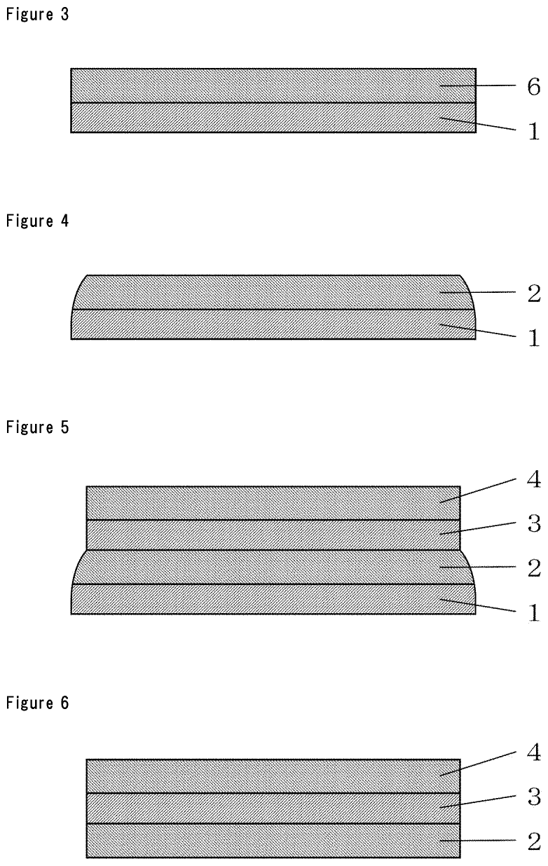

The medical tape of the present invention includes the release portion, the adhesion portion, the elastic base member portion, and the support portion in this order from a lower layer. In the manufacturing process, a solution polymer used as a material of the elastic base member portion is applied on the upper layer of the adhesion portion to form a film, the elastic base member portion is provided with an elastic function, and the support portion is laminated on the upper layer of the elastic base member portion after or before the completion of the forming shrinkage. Consequently, as illustrated in FIG. 3, the elastic base member portion has a trapezoidal shape with the upper base shorter than the lower base in association with the forming shrinkage of the solution polymer. Comparing with the residual shrink force of the elastic base member portion manufactured by the prior art in which the film formation and lamination with the support portion are simultaneously performed, the lower base portion of the elastic base member portion has an equivalent residual shrink force, but the residual shrink force on the upper base portion is reduced or eliminated. Note that the residual shrink force existing in the lower base portion of the elastic base member portion can be reduced or eliminated by removing the release portion when in use of the support portion-bonded medical tape.

A timing to laminate the support portion on the upper layer of the elastic base member portion is preferably after the completion of the forming shrinkage in which the elastic base member portion has a trapezoidal shape with the upper base shorter than the lower base in association with the forming shrinkage of the solution polymer and no residual shrink force is generated on the upper base portion of the elastic base member portion even if the support portion is laminated thereon. However, a time before the completion of the forming shrinkage may be selected if appropriate by taking materials of the support portion and the solution polymer to be selected, manufacturing equipment, production efficiencies, and the like into consideration. Even if the time before the completion of the forming shrinkage is selected and the residual shrink force is generated on the upper base portion of the elastic base member portion, the residual shrink force can be reduced compared with the prior art in which the polyurethane film formation and lamination with the support portion are simultaneously performed. Thus, the timing to laminate the support portion on the upper layer of the elastic base member portion can be before or after the completion of the forming shrinkage and is not specifically limited as long as the effect of the present invention can be provided.

In the medical tape of the present invention, the adhesion portion is provided on the upper layer of the release portion that is held horizontally, a solution polymer used as a material of the elastic base member portion is applied on the upper layer of the adhesion portion to form a film, and the elastic base member portion is provided with an elastic function. At this time, as illustrated in FIG. 3, the elastic base member portion is configured to have the cross-sectional surface in a trapezoidal shape with the upper base shorter than the lower base in association with the forming shrinkage of the solution polymer, and then the support portion is laminated on the upper layer of the elastic base member portion. Accordingly, the residual shrink force existing in the support portion-bonded medical tape can be reduced by removing the release portion when in use of the support portion-bonded medical tape. Note that the degree of the residual shrink force to be reduced is preferably a complete elimination. However, the degree of the residual shrink force as a cause is as insignificant as to be described as of a level of magnifying lens or microscope, not as of a level of naked eye, and thus, the residual shrink force to be reduced may be insignificant and the effect of the present invention can be sufficiently provided even if it is insignificant.

While examples of the present invention will now be described in details with reference to the accompanying drawings, the present invention is not limited thereto. In appropriately selecting and combining the materials that constitute the support portion-bonded medical tape and in the manufacturing process, the present invention is based on applying a mixed liquid (a urethane resin liquid and a crosslinking agent liquid) serving as a raw material of a polyurethane film to become the elastic base member portion later on the upper layer of the adhesion portion, after the completion of the forming shrinkage (FIG. 3), laminating the support portion on the upper layer of the elastic base member portion (FIG. 4), and removing the release portion when in use of the support portion-bonded medical tape (FIG. 5), thereby releasing the residual shrink force existing in the support portion-bonded medical tape, reducing the force that continuously shrinks the skin due to the residual shrink force of the support portion-bonded medical tape, and alleviating the persistent skin irritation during the application duration caused by the residual shrink force. The present invention needs to reduce the residual shrink force twice when the support portion-bonded medical tape is manufactured and when it is used.

A support portion-bonded medical tape of a first embodiment of the present invention illustrated in FIG. 3 to FIG. 6 is configured of an elastic base member portion 1, an adhesion portion 2, a support portion 3, and a release portion 4. The elastic base member portion 1 is film-formed by a mixed liquid (a urethane resin liquid and a crosslinking agent liquid) as a raw material of the elastic base member portion and has the elastic function. The adhesion portion 2 has the function to apply and hold the elastic base member portion 1 on a skin. The release portion 4 has the function to protect the adhesion portion. The support portion 3 has the function to hold the elastic base member portion 1.

FIG. 3 is a drawing illustrating the manufacturing process of the support portion-bonded medical tape according to the first embodiment of the present invention, is a schematic cross-sectional view illustrating a state of a laminating process of the release portion, the adhesion portion and the elastic base member portion, and is an explanatory diagram illustrating a state in which the elastic base member portion has a trapezoidal shape with the upper base shorter than the lower base in association with the forming shrinkage of the mixed liquid (the urethane resin liquid and the crosslinking agent liquid) serving as the raw material of the elastic base member portion. In the support portion-bonded medical tape according to the first embodiment, since the residual shrink force existing in the support portion-bonded medical tape is released by removing the release portion when in use of the support portion-bonded medical tape, the mixed liquid (the urethane resin liquid and the crosslinking agent liquid) serving as the raw material of the polyurethane film that turns into an elastic base member portion 1 later was applied on an upper layer of an adhesion portion 2 in the manufacturing process to form a film. As illustrated in FIG. 3, the elastic base member portion 1 after the film formation has the cross-sectional surface in a trapezoidal shape with the upper base shorter than the lower base in association with the forming shrinkage of the mixed liquid (the urethane resin liquid and the crosslinking agent liquid) serving as the raw material of the elastic base member portion. Note that the reference numeral 1 denotes the elastic base member portion, the reference numeral 2 denotes the adhesion portion, and the reference numeral 4 denotes the release portion.

FIG. 4 is a schematic cross-sectional view illustrating the support portion-bonded medical tape according to the first embodiment of the present invention. The support portion is laminated on the trapezoidal elastic base member portion with the upper base shorter than the lower base, which is illustrated in FIG. 3, and then the support portion-bonded medical tape is completed. As illustrated in FIG. 3, the support portion-bonded medical tape has a trapezoidal shape with the upper base shorter than the lower base in association with the forming shrinkage of the mixed liquid (the urethane resin liquid and the crosslinking agent liquid) serving as the raw material of the elastic base member portion, the film formation is completed, and then as illustrated in FIG. 4, the support portion 3 with the same length as that of the elastic base member portion 1 is laminated on the upper layer of the elastic base member portion 1 after the forming shrinkage is completed. At this time, the residual shrink force exists in the lower base portion of the elastic base member portion 1. Note that the reference numeral 1 denotes the elastic base member portion, the reference numeral 2 denotes the adhesion portion, the reference numeral 3 denotes the support portion, and the reference numeral 4 denotes the release portion.

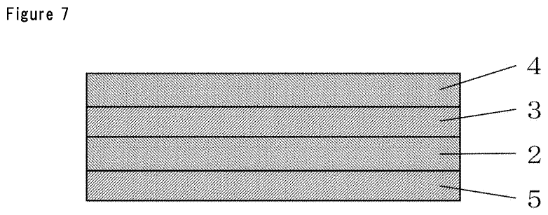

FIG. 5 and FIG. 6 are schematic cross-sectional views illustrating a state when in use of the medical tape according to the first embodiment of the present invention. The support portion-bonded medical tape is used in the following sequence: 1) remove the releasing portion; 2) apply the tape on the skin; and 3) remove the support portion. FIG. 5 illustrates a state where the release portion 4 is removed and the residual shrink force having existed in the lower base portion of the elastic base member portion 1 is eliminated without limit. FIG. 6 illustrates a state where the adhesion portion 2 is applied on a skin 5 and the support portion 3 is removed in the state illustrated in FIG. 5. Note that the reference numeral 1 denotes the elastic base member portion, the reference numeral 2 denotes the adhesion portion, the reference numeral 3 denotes the support portion, and the reference numeral 5 denotes the skin.

In this support portion-bonded medical tape, the adhesion portion is provided on the upper layer of the release portion, and the solution polymer used as a material of the elastic base member portion is applied on the upper layer of the adhesion portion to form a film in the manufacturing process. At this time, the elastic base member portion has the cross-sectional surface in a trapezoidal shape with the upper base shorter than the lower base in association with the forming shrinkage of the solution polymer (FIG. 3). In the trapezoidal elastic base member portion, the residual shrink force associated with the forming shrinkage of the solution polymer is generated on the lower base of the elastic base member portion attached on the adhesion portion. On the other hand, since the upper base of the elastic base member portion is not laminated at this moment, it is not attached and shrinks freely, and the residual shrink force is in a state of decreasing from the lower base of the elastic base member portion attached on the adhesion portion toward the upper base of the elastic base member portion in the film thickness direction. Therefore, since there is a difference in the residual shrink force associated with the forming shrinkage of the solution polymer between the lower base of the elastic base member portion in contact with the adhesion portion and the upper base of the elastic base member portion, there is a difference in length and the elastic base member portion has a trapezoidal shape. The residual shrink force on the upper base of the elastic base member portion can be said to be reduced as compared with that on the lower base of the elastic base member portion in contact with the adhesion portion. Then, after the completion of the forming shrinkage of the solution polymer, the support portion with the same length as that of the upper base of the elastic base member portion is laminated on the upper base of the elastic base member portion (FIG. 4), and the support portion-bonded medical tape is completed.

The support portion-bonded medical tape manufactured as described above is used in the following sequence: 1) remove the releasing portion; 2) apply the tape on the skin; and 3) remove the support portion. In the support portion-bonded medical tape, the release portion is removed first. Then, the elastic base member portion and the adhesion portion of the support portion-bonded medical tape shrink and have the same length as that of the support portion (FIG. 5). This is because the adhesion portion follows the elasticity of the elastic base member portion by removing the release portion, the residual shrink force generated on the lower base of the elastic base member portion attached on the adhesion portion is released, and the residual shrink force existing in the elastic base member portion is reduced. The length of the elastic base member portion and the adhesion portion of the support portion-bonded medical tape shrinks and shortens for the amount of the residual shrink force that has been released, and then the elastic base member portion and the adhesion portion have the same length as that of the support portion. Then, the tape is applied on the skin, and the support portion is removed. The support portion and the upper base of the elastic base member portion are laminated after the completion of the forming shrinkage of the solution polymer. Therefore, the residual shrink force associated with the forming shrinkage of the solution polymer is in a reduced (eliminated) state on the upper base of the elastic base member portion attached on the support portion. Accordingly, a clear change such as a shrinkage of the elastic base member portion does not occur even though the support portion is removed.

In the support portion-bonded medical tape, the residual shrink force of the support portion-bonded medical tape can be reduced again by removing the release portion before the tape is applied on the skin, and the support portion-bonded medical tape can be applied on a skin in a state where the residual shrink force is reduced. Accordingly, the residual shrink force is reduced twice when the support portion-bonded medical tape is manufactured and when it is used, and the force that continuously shrinks the skin caused by the residual shrink force of the support portion-bonded medical tape is reduced, thereby ensuring alleviating the persistent skin irritation during the application duration. Accordingly, in the support portion-bonded medical tape of the present invention, by introducing the means for reducing the force that continuously shrinks the skin caused by the residual shrink force associated with the forming shrinkage of the conventional support-bonded medical tape to a medical tape as a function, it is possible to apply the medical tape on the skin in the state where the residual shrink force of the support portion-bonded medical tape is reduced when in use of the support portion-bonded medical tape, reduce the force that continuously shrinks the skin due to the residual shrink force of the portion-bonded medical tape medical tape, and alleviate the persistent skin irritation during the application duration caused by the residual shrink force. This ensures an application on the skin that exhibits an excessive reaction even when a degree of residual shrink force as a cause is an insignificant irritation so as to be described as of a level of magnifying lens or microscope, not as of a level of naked eye, thereby ensuring providing a medical tape, an adhesive skin patch, and the like of low irritation.

Specifically, in the first embodiment of the present invention, in the manufacturing process, the release portion 4 was prepared with a release sheet formed by coating a release agent on a surface of a high-quality paper, and the adhesion portion 2 was formed by applying an acrylic adhesive on the upper layer of the release portion 4. The polyurethane film as the elastic base member portion 1 was obtained by applying the mixed liquid (the urethane resin liquid and the crosslinking agent liquid) serving as the raw material of the elastic base member portion that turned into the elastic base member portion 1 later on the upper layer of the adhesion portion 2 and then hardening the mixed liquid to form a film, and the film formation was completed. The elastic base member portion had a trapezoidal shape with the upper bas shorter than the lower base in association with the forming shrinkage of the mixed liquid (the urethane resin liquid and the crosslinking agent liquid) serving as the raw material of the elastic base member portion. While it is not illustrated, the mixed liquid (the urethane resin liquid and the crosslinking agent liquid) serving as the raw material of the elastic base member portion 1 was thinly applied on the lower layer of the support portion 3 and laminated on the upper base of the elastic base member portion 1 in a state of having a role of an adhesion after the forming shrinkage was completed as a time when the residual shrink force was not generated on the upper base portion of the elastic base member portion Leven if the support portion was laminated.

While in the first embodiment, the mixed liquid (the urethane resin liquid and the crosslinking agent liquid) serving as the raw material of the polyurethane film is used as the material of the elastic base member portion, any solution polymer material that can be applied on the upper layer of the adhesion portion and laminated with the support portion after it is hardened to form a film may be used as the material of the elastic base member portion. If the solution polymer material is any solution polymer materials that can provide functions appropriate for respective usages, such as covering and fixing a gauze, a pad, and the like, fixing a surgical tool, such as an indwelling needle and a catheter, and covering and protecting wounds or skin, another new material and the like may be used, or different solution polymer materials may be mixed or laminated. The material is not specifically limited as long as the effect of the present invention can be provided. While the acrylic adhesive is used for the material of the adhesion portion, the material of the adhesion portion may be any adhesive that has a function to apply and hold the elastic base member portion on the skin, has a function to follow the elastic function of the elastic base member portion, and is usable for medical treatment, and the adhesive may be applied partly or entirely to correspond to the usage, and thus, the material and the shape are not restricted. While as the material of the support potion, an inelastic plastic film that has a function to support the elastic base member portion is used in a state of a single sheet that is not physically separated, the support portion may have any stiffness as long as the material of the support potion has a function to support the elastic base member portion, for example, the material may be divided at the center into two pieces or divided with a wavy notch. In addition, the support portion and the upper layer thereof may have another function, and as a result, the support portion may be peeled off partially from the elastic base member portion when in use as long as it holds the elastic base member portion in a state in which the elastic base material can be applied after the release sheet is removed. The material and the shape are not restricted to correspond to the usage.

In the first embodiment, a time after the forming shrinkage was completed as a time when the forming shrinkage was completed, the elastic base member portion had a trapezoidal shape with the upper base shorter than the lower base in association with the forming shrinkage of the mixed liquid (the urethane resin liquid and the crosslinking agent liquid) serving as the raw material of the polyurethane film, as illustrated in FIG. 3, and the residual shrink force was not generated on the upper base portion of the elastic base member portion even when the support portion was laminated was selected for a timing to laminate the support portion on the elastic base member portion. While it is not illustrated, the mixed liquid (the urethane resin liquid and the crosslinking agent liquid) serving as the raw material of the polyurethane film was ultra-thinly applied between the support portion and the elastic base member, and the support portion and the elastic base member portion were laminated in a state of having a role of an adhesion. However, a time before the completion of the forming shrinkage may be selected to laminate the support portion by taking materials of the elastic base member portion and the support portion to be used, manufacturing equipment, production efficiencies, and the like into consideration. Even if the time before the completion of the forming shrinkage is selected and the residual shrink force is generated on the upper base portion of the elastic base member portion, the residual shrink force can be reduced compared with the prior art in which the film formation of the elastic base member portion and the lamination on the support portion are simultaneously performed. Note that the degree of the residual shrink force as a cause of the persistent skin irritation during the application duration is as insignificant as to be described as of a level of magnifying lens or microscope, not as of a level of naked eye, and thus, the residual shrink force to be reduced may be insignificant and the effect of the present invention can be sufficiently provided even if it is insignificant. Note that “before or after the completion of the forming shrinkage” here refers to the time before or after the solution polymer used as the material of the elastic base member portion is applied on the upper layer of the adhesion portion, and the forming shrinkage of the solution polymer is completed in the process of forming a film. Specifically, it refers to a period from the time when the residual shrink force existing between the support portion and the elastic base member portion can be theoretically determined to be reduced compared with the residual shrink force existing between the support portion and the elastic base member portion of the support-bonded medical tape which is manufactured by the prior art even if the forming shrinkage of the solution polymer begins and the support portion is laminated on the upper layer thereof (before the completion of the forming shrinkage) to the time when no residual shrink force can be theoretically determined to be generated between the support portion and the elastic base member portion even if the forming shrinkage of the solution polymer is completed and the support portion is laminated on the upper layer thereof (after the completion of the forming shrinkage). While the release sheet formed by coating the release agent on the surface of the high-quality paper is used for the material of the release portion, the material may be optional and the material and the shape are not restricted to correspond to the usage, design, convenience, and the like as long as the material of the release portion has the function to protect the adhesion portion.

The medical tape of the present invention illustrated in FIG. 3 to FIG. 6 all has a basic structure of the present invention. Such a configuration ensures applying the medical tape on the skin in the state where the residual shrink force of the support portion-bonded medical tape is reduced, reducing the force to continuously shrink the skin caused by the residual shrink force of the support portion-bonded medical tape, and alleviating the persistent skin irritation during the application duration caused by the residual shrink force, when in use of the support portion-bonded medical tape, by introducing the means for reducing the force to continuously shrink the skin caused by the residual shrink force associated with the forming shrinkage of the conventional support-bonded medical tape to the medical tape as a function. This ensures an application on the skin that exhibits an excessive reaction even when a degree of residual shrink force associated with the forming shrinkage as a cause is an insignificant irritation so as to be described as of a level of magnifying lens or microscope, not as of a level of naked eye, thereby ensuring providing a medical tape, an adhesive skin patch, and the like of low irritation. Preventions of inflammation in sebaceous glands or sweat glands, inflammation in hair roots and surrounding tissues, medical instrument pressure-related wounds caused by poor blood circulation of cells of skin, capillary blood vessels, and the like caused by pressure, and the like can be expected.

While the example where the medical tape has a shape of rectangular has been described in the first embodiment above, any shape can be selected for the shape of the medical tape to correspond to the usage. A rolled shape and a sheet shape are possible, and a shape that fits each part of body is also possible. While the medical tape is basically configured of the release portion, the adhesion portion, the elastic base member portion, and the support portion, a portion not having any functions that each portion of the medical tape has may be included in a part of the medical tape and a gauze, a pad, and the like may be provided to correspond to the usage, design, convenience, and the like.

Note that while the present invention is based on the application on a skin in a closely contacted manner, the medical tape may be applied via a protecting material and the like for protecting the skin from the medical adhesive and the like in some cases.

While all the residual shrink force is preferred to be eliminated, a state where it is slightly reduced is also possible. This is because, as described in the paragraphs [0017] to [0024], the degree of residual shrink force that this patent is solving is not of a level of naked eye, but is of a level of magnifying lens or microscope. In this description, the process from the manufacturing of the medical tape to the application is described, and “when in use” and “during application duration” are defined as follows. “When in use” represents the duration from removing the release portion, applying on a skin, until removing the support following the procedure of using the medical tape. “During application duration” represents the duration where the medical tape is continuously applied.

Note that the function of the medical tape according to the present invention is effectively applied to a medical tape aiming to be applied to a human body. For example, since a skin irritation hypersensitivity patient severely reacts to even an insignificant skin irritation, the use of medical tapes containing percutaneously absorbable drugs is postponed in some cases. However, since it is possible to alleviate the insignificant skin irritation by using the medical tape according to the present invention, the medical tape according to the present invention can be used as a patch by blending percutaneously absorbable drugs in the adhesive portion. Providing a pad to the medical tape of the present invention ensures a usage as a treatment product.

### INDUSTRIAL APPLICABILITY

The support portion-bonded medical tape according to the present invention has an extremely high effect of alleviating the persistent skin irritation during the application duration caused by the residual shrink force associated with the forming shrinkage of the support-bonded medical tape so as to be applicable on the skin that exhibits an excessive reaction even with an insignificant irritation. In addition, preventions of a skin trouble, a medical instrument pressure-related wound, and the like caused by the persistent skin irritation during the application duration can be expected. Furthermore, it is possible to provide a low-irritation medical tape, patch or the like. Therefore, the medical tape according to the present invention will greatly contribute to the medical industries.

### REFERENCE SIGNS LIST

1 elastic base member portion
2 adhesion portion
3 support portion
4 release portion
5 skin

1. A method for manufacturing a support portion-bonded medical tape, wherein
the medical tape includes an elastic base member portion film-formed of a solution polymer and having an elastic function, an adhesion portion having a function to apply and hold the elastic base member portion on a skin, a release portion having a function to protect the adhesion portion, and a support portion having a function to support the elastic base member portion, the support portion, the elastic base member portion, the adhesion portion, and the release portion being provided in this order from an upper layer, the method comprises, in a manufacturing process of the medical tape,
applying the solution polymer used as a material for the elastic base member portion to an upper layer of the adhesion portion, and laminating the support portion on an upper layer of the elastic base member portion before or after completion of forming shrinkage, and
the method comprises reducing a residual shrink force existing in the support portion-bonded medical tape by removing the release portion at a time of use.
